(19) United States
(12) Patent Application Publication
(10) Pub. No.: US 2014/0025051 A1
(43) Pub. Date: Jan. 23, 2014

(54) APPARATUS FOR OPTICAL SURGERY AND METHOD FOR CONTROLLING SAME

(71) Applicant: Kwang Chon Ko, Gyeonggi-do (KR)

(72) Inventor: Kwang Chon Ko, Gyeonggi-do (KR)

(73) Assignee: LUTRONIC CORPORATION, Gyeonggi-do (KR)

(21) Appl. No.: 14/007,280

(22) PCT Filed: Apr. 25, 2011
(86) PCT No.: PCT/KR11/02979

§ 371 Date: Sep. 24, 2013

(30) Foreign Application Priority Data
Mar. 25, 2011 (KR) 10-2011-0026739
Mar. 28, 2011 (KR) 10-2011-0027501

(51) Int. Cl. A61B 18/22

(52) U.S. Cl. CPC A61B 18/22
USPC 606/11

(57) ABSTRACT

The present invention relates to an apparatus for optical surgery and to the method for controlling same, and provides the apparatus for optical surgery and a method for controlling same, comprising: a cutting open-energy source for generating the cutting open-energy for cutting open a surgery area; a hemostasis energy source for generating energy for hemostasis, which is formed separately from the cutting open-energy source, for stopping bleeding at the cut open surgery area; a hand piece, which is connected to the cutting open-energy source and the hemostasis energy source, for providing the cutting open-energy and the hemostasis energy to the surgery area; and a control part for controlling the cutting open-energy and the hemostasis energy, which are provided through the hand piece.

### TECHNICAL FIELD

The present invention relates to an optical surgery apparatus and a method of controlling the same and, more particularly, to an optical surgery apparatus capable of incising tissue within the body and stopping the flow of blood from the incised part and a method of controlling the same.

### BACKGROUND ART

Recently, a technique for performing medical treatment in such a way as to radiate light toward the body and change the state of a tissue by means of light energy absorbed by the tissue of the body is widely used.

A product using a laser as a light source is commonly used as a medical treatment apparatus using such light. Lasers having various wavelength bands, such as an Nd:YAG laser, a KTP laser, an ER:YAG laser, a CO2 laser, an Ho:YAG laser, a ruby laser, and an alexandrite laser, are being used. The product is widely applied to purposes for hair removal and skin management and surgical and internal operations.

In particular, an internal operation on tissue within the body is replaced with an optical surgery apparatus for radiating a laser through optical fibers, instead of a surgical operation performed by inserting various existing operation tools into the body, and various surgical operations, such as a surgical operation for incising tissue within the body using laser energy, are used in the internal operation.

Such an optical surgery apparatus for incising tissue within the body is used in a surgical operation for incising a tissue using light having continuous waves and stopping the flow of blood from the incised tissue. However, there are problems in that tissue adjacent to an incised tissue is damaged because heat energy is accumulated when a light source having continuous waves is used and the time taken for a surgical operation is long. In order to solve the problems, a surgical operation for incising tissue within the body by applying an impulse to the tissue within the body using light having a pulse wave is recently being used. However, an optical surgery apparatus using such a pulse wave is disadvantageous in that the flow of blood from an incised part is not properly stopped.

### DISCLOSURE

### Technical Problem

The present invention provides an optical treatment apparatus capable of incising tissue within the body using light having pulse waves and easily stopping the flow of blood from an incised part and a method of controlling the same.

### Technical Solution

The above-described conventional problems may be solved by an optical surgery apparatus, including an incision energy source for generating energy for incision for incising a surgical site, a hemostasis energy source formed separately from the incision energy source, for generating energy for hemostasis for stopping the flow of blood from the incised surgical site, a handpiece connected with the incision energy source and the hemostasis energy source, for providing the energy for incision and the energy for hemostasis to the surgical site, and a control unit for controlling the energy for incision and the energy for hemostasis supplied through the handpiece.

Here, the incision energy source may be configured to generate light for incision, and the hemostasis energy source may be configured to generate radio frequency electronic energy for hemostasis.

Or, the incision energy source may be configured to generate light for incision, and the hemostasis energy source may be configured to generate light for hemostasis having a different wavelength from the light for incision.

In one embodiment, there is provided an optical surgery apparatus, including a light radiation unit for radiating light, generated from a light generation unit, to a surgical site within a body, a radio frequency supply unit installed adjacent to the end of the light radiation unit, for providing radio frequency electronic energy to a part to which the light is radiated, an insulating unit formed to surround part of the radio frequency supply unit, and a control unit for controlling driving of the light radiation unit and the radio frequency supply unit.

The light radiation unit radiates incision light for incising tissue within the body, and the radio frequency supply unit supplies the radio frequency electronic energy for stopping the flow of blood from the incised tissue.

The radio frequency supply unit includes a thin pipe in which a hole is formed in a length direction, and the light radiation unit is inserted into and installed in the hole.

A mono-polar type radio frequency electrode may be formed at the front end of the radio frequency supply unit, and the optical surgery apparatus may be configured to further include an external electrode having a different polarity from the radio frequency electrode and formed to be attached to a surface of the body.

Or, a plurality of radio frequency electrodes may be formed at the front end of the radio frequency supply unit, some of the plurality of radio frequency electrodes may form positive electrodes and the remaining radio frequency electrodes may form negative electrodes.

Meanwhile, the above-described conventional problems may also be solved by an optical surgery apparatus, including a light generation unit for generating incision light and hemostasis light having a different wavelength from the incision light, a light radiation unit for selectively radiating the incision light and the hemostasis light, generated from the light generation unit, to a surgical site, and a control unit for controlling a radiation pattern of the light radiated through the light radiation unit.

The control unit controls the light radiation unit so that the incision light having greater output than the hemostasis light is radiated.

Here, each of the incision light and the hemostasis light is pulse light that is periodically intermitted for a predetermined time, and the hemostasis light may be radiated so that it is intermitted for a time shorter than a time of the incision light.

Or, the incision light may be pulse light intermitted in a predetermined period, and the hemostasis light may be continuous light.

### Advantageous Effects

In accordance with the present invention, there are advantages in that the time taken for a surgical operation can be reduced and convenience of a person who undergoes a surgical operation can be improved because a surgical operation is performed by incising tissue within the body using incision light and stopping the flow of blood from the incised part using radio frequency electronic energy or hemostasis light having a different wavelength from the incision light.

### DESCRIPTION OF DRAWINGS

FIG. 1 is a perspective view showing an optical surgery apparatus in accordance with a first embodiment of the present invention,

FIG. 2 is a block diagram schematically showing the construction of the optical surgery apparatus of FIG. 1,

FIG. 3 is a cross-sectional view showing a section of a handpiece of FIG. 1,

FIG. 4 is a cross-sectional view showing another example of a section of the handpiece of FIG. 1,

FIG. 5 is a cross-sectional view showing a state in which surgery is performed using the handpiece of FIG. 3,

FIG. 6 is a flowchart illustrating a method of controlling the optical surgery apparatus of FIG. 1,

FIG. 7 is a front view showing the end of the handpiece of an optical surgery apparatus in accordance with a second embodiment of the present invention,

FIG. 8 is a cross-sectional view showing a state in which surgery is performed using the handpiece of FIG. 7,

FIG. 9 is a perspective view showing an optical surgery apparatus in accordance with a third embodiment of the present invention,

FIG. 10 is a block diagram schematically showing the construction of the optical surgery apparatus of FIG. 9,

FIG. 11 is a graph showing pulse forms of first light and second light,

FIG. 12 is a graph showing a radiation pattern of light that is radiated from a light radiation unit of FIG. 11,

FIG. 13 is a flowchart illustrating a method of controlling the optical surgery apparatus of FIG. 9,

FIG. 14 is a perspective view showing an optical surgery apparatus in accordance with a fourth embodiment of the present invention.

### MODE FOR INVENTION

Hereinafter, preferred embodiments of the present invention are described in more detail with reference to the accompanying drawings. In the following embodiments, it is to be noted that elements are simplified in order to clearly describe the present invention, the present invention is not limited to the embodiments, and the present invention can be implemented by adding various additional apparatuses or by means of applications and design.

A description of the present embodiment is based on the accompanying drawings. Furthermore, expressions that describe locations and connection relationships between elements include a case where corresponding elements are directly coupled as well as a case where the corresponding elements are indirectly coupled.

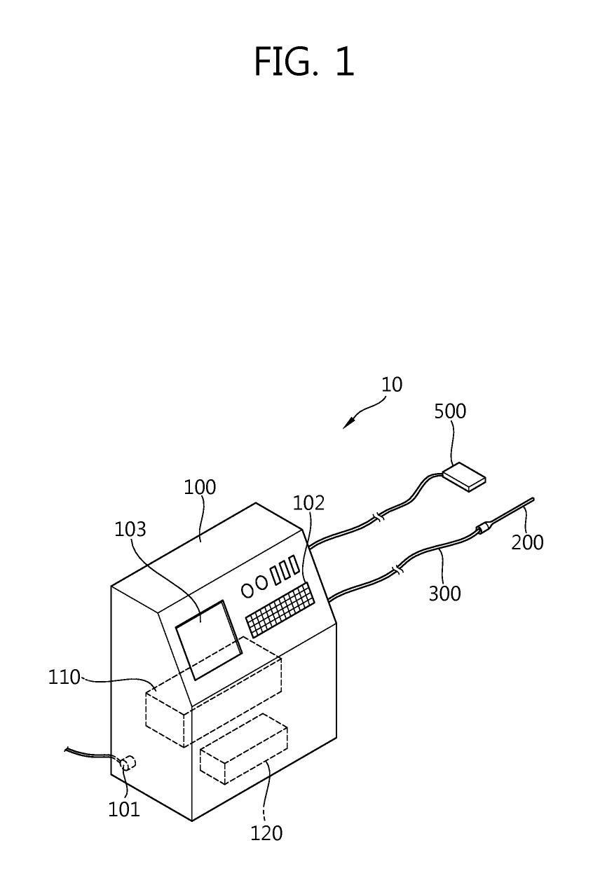

FIG. 1 is a perspective view showing an optical surgery apparatus in accordance with a first embodiment of the present invention. As shown in FIG. 1, the optical surgery apparatus 10 in accordance with the present embodiment includes a main body 100, a handpiece 200, and a cable 300 coupling the main body 100 and the handpiece 200.

The main body 100 is equipped with a power supply unit 101 capable of being supplied with an external power source. A control panel 102 for manipulating contents of driving of the optical surgery apparatus 10 and a display 103 for displaying the contents of driving to a user can be installed on the outside of the main body. Furthermore, a light generation unit 110 for generating light for a surgical operation and a radio frequency generation unit 120 for generating radio frequency electronic energy are provided on the inner side of the main body 100.

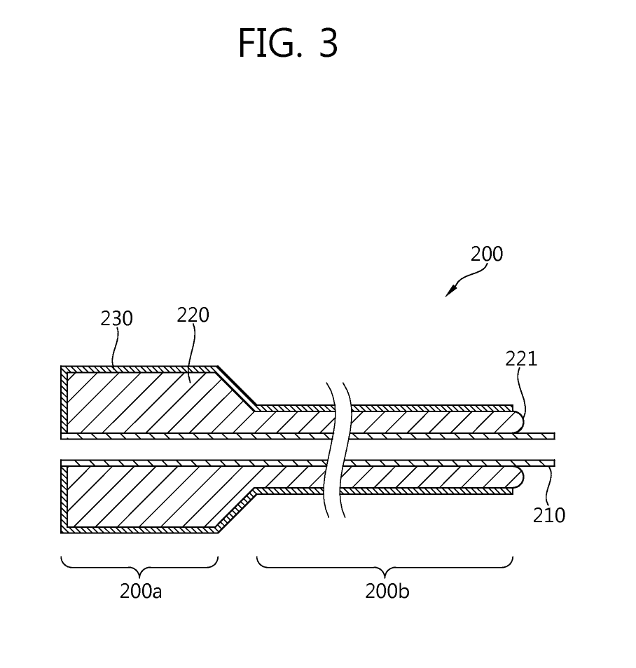

The handpiece 200 is an element that is inserted into the body when a surgical operation is performed in order to perform the surgical operation. The handpiece 200 is configured to be grasped by a user. The handpiece 200 includes a grasp part 200a that can be manipulated in the state in which the grasp part is grasped by the hand of a user and an insertion unit 200b inserted into the body (refer to FIG. 3). Accordingly, the insertion unit 200b is configured to have a thin pipe so that the insertion unit can be inserted into the interior of the body. Furthermore, various elements for surgical operations are installed at the end of the handpiece 200 corresponding to the insertion unit 200b.

In the concrete, a light path along which light travels is formed within the handpiece 200, and a light radiation unit 210 for radiating light to a surgical site is formed at the front of the handpiece 200. The handpiece 200 further includes a radio frequency supply unit 220 for supplying radio frequency electronic energy to a surgical site.

In the present embodiment, although not separately included, a photographing apparatus or a lighting apparatus for capturing an image of a surgical site and various devices for performing various surgical operations may be installed in the cannula.

Meanwhile, the cable 300 is formed between the main body 100 and the handpiece 200 and configured to include a light transfer unit 310 and a radio frequency transfer unit 320. The light transfer unit 310 forms a path along which light generated from the light generation unit 110 of the main body 100 travels to the light radiation unit 210 of the handpiece 200. The radio frequency transfer unit 320 forms a path along which a radio frequency generated from the radio frequency generation unit 120 of the main body 100 is provided to the radio frequency supply unit 220 of the handpiece 200.

The light transfer unit 310 and the radio frequency transfer unit 320 may be embedded and installed within one cable 300 or may be configured as separate elements so that they are easily replaced.

Meanwhile, as shown in FIG. 1, an additional external electrode 500 connected with the radio frequency generation unit 120 of the main body 100 can be further included. The radio frequency supply unit 220 included in the handpiece 200 includes a mono-polar type electrode, and the external electrode 500 forms an electrode having different polarity from the electrode included in the radio frequency supply unit 220. The external electrode 500 is formed in such a way as to be attached to the outside of the body. Accordingly, when radio frequency electronic energy is generated from the radio frequency generation unit 120 during a surgical operation, the radio frequency electronic energy is supplied to the body through the radio frequency supply unit 220 placed inside the body and the external electrode 500 attached to the outside of the body.

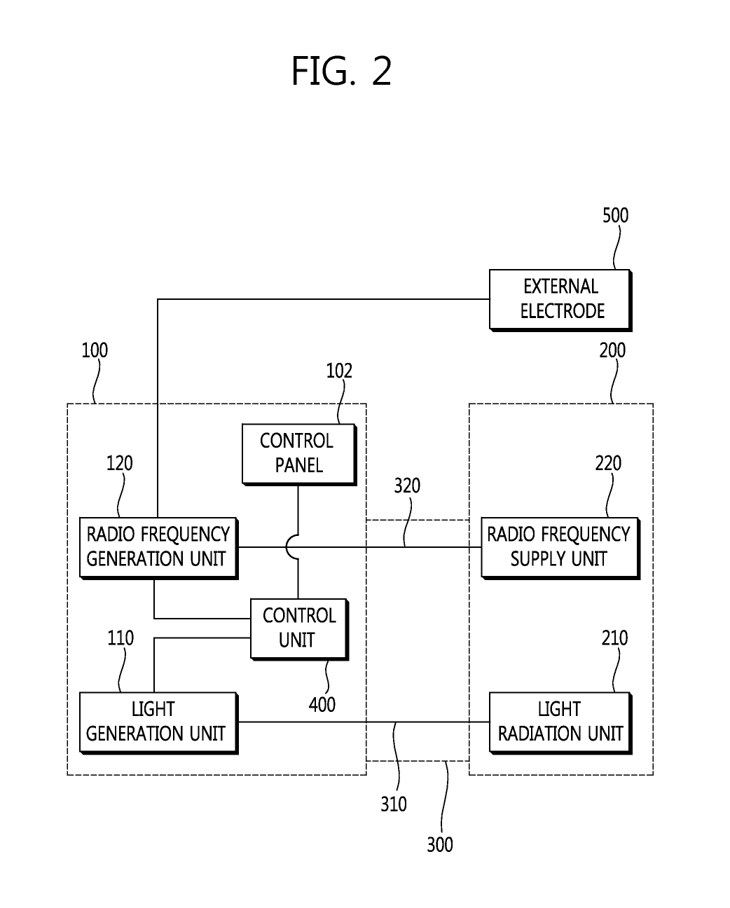

FIG. 2 is a block diagram schematically showing the construction of the optical surgery apparatus of FIG. 1. Hereinafter, the construction of the optical surgery apparatus in accordance with the present embodiment is described in more detail with reference to FIG. 2.

The light generation unit 110 of the present embodiment is installed within the main body and is a device for generating light used when a surgical operation is performed. In the present embodiment, the light generation unit is an incision energy source, and light generated from the light generation unit is used as light for incision. Accordingly, light generated from the light generation unit is radiated to a surgical site, thus providing energy for incision to the surgical site.

The light generation unit 110 can be configured using various types of light sources. In the present embodiment, for example, as shown in FIG. 2, the light generation unit 110 is equipped with a laser resonator for oscillating laser light. In the concrete, the light generation unit 110 includes a resonator using Nd:YAG as a medium and generates laser light having a wavelength of 1444 nm. However, the present invention is not limited to the type of media or the wavelength of the light generation unit, and light having different wavelengths can be used using various media depending on contents that will undergo a surgical operation.

Meanwhile, the light transfer unit 310 that forms a light path is installed on one side of the light generation unit 110. Although not separately shown, various optical elements can be included between the light generation unit 110 and the light transfer unit 310 so that light generated from the light generation unit 110 enters one end of the light transfer unit 310 when the light is generated. Furthermore, light that travels along the light transfer unit 310 is radiated to a surgical site through the light radiation unit 210 of the handpiece 200.

Meanwhile, the radio frequency generation unit 120 is installed within the main body 100 and is configured to generate radio frequency electronic energy using a power source received from the power supply unit 101. In the present embodiment, the radio frequency generation unit 120 is a hemostasis energy source, and radio frequency electronic energy generated from the radio frequency generation unit is provided to a surgical site and used as energy for hemostasis.

In the concrete, the radio frequency generation unit 120 is electrically connected with the external electrode 500 and the radio frequency supply unit 220 of the handpiece 200. Here, one of the radio frequency supply unit 220 and the external electrode 500 is a positive electrode, and the other thereof forms a negative electrode. Accordingly, in the state in which the radio frequency supply unit 220 of the handpiece 200 is placed inside the body and the external electrode 500 is attached to the outside of the body, the positive and negative electrodes form a circuit using the body as a medium and thus supply radio frequency electronic energy to the interior of the body.

Here, the radio frequency generation unit 120 may be configured to generate radio frequency electronic energy having various frequency bands depending on a user setting or may be configured to generate radio frequency electronic energy having a specific frequency band.

Meanwhile, the optical surgery apparatus in accordance with the present embodiment includes a control unit 400 for controlling various elements. The control unit 400 can control contents of driving of the optical surgery apparatus based on contents set by a user through the control panel 102, based on contents manipulated by a user if a manipulation unit that can be separately manipulated by a user during a surgical operation is provided, or in a mode stored in its memory (not shown).

For example, the control unit 400 can control the output of light and a pulse waveform of light generated from the light generation unit 110 and control the output of radio frequency electronic energy and a frequency of radio frequency electronic energy generated from the radio frequency generation unit 120 by controlling circuits that are connected with the light generation unit 110 and the radio frequency generation unit 120. Furthermore, the control unit 400 may control the time when light is radiated from the light radiation unit 210 and radiation duration for which the light is radiated and may control the time when radio frequency electronic energy is supplied by the radio frequency supply unit 220 and radiation duration for which the radio frequency electronic energy is radiated.

In addition, although not shown, if the optical surgery apparatus includes an additional photographing unit or lighting unit, etc., the control unit may control the additional photographing unit or lighting unit.

FIG. 3 is a cross-sectional view showing a section of the handpiece of FIG. 1. The construction of the optical surgery apparatus in accordance with the present embodiment is described in more detail with reference to FIG. 3.

As described above, the handpiece 200 includes the radio frequency supply unit 220 and the light radiation unit 210.

As shown in FIG. 3, the radio frequency supply unit 220 is formed in the body of the handpiece 200, and a hole is formed within the radio frequency supply unit 220. A portion of the radio frequency supply unit 220 corresponding to the grasp part 200a has a pipe form having a relatively thick diameter. Furthermore, a portion of the radio frequency supply unit 220 corresponding to the insertion unit 200b has a thin pipe form having a relatively thin diameter.

The radio frequency supply unit 220 is made of metal materials having excellent conductivity. In the present embodiment, the radio frequency supply unit 220 is made of Steel Use Stainless (SUS) material. Furthermore, the radio frequency supply unit 220 is connected with the radio frequency transfer unit 320 and is supplied with radio frequency electronic energy generated from the radio frequency generation unit 120.

Here, insulating materials are formed on the exterior of the radio frequency supply unit 220 (refer to FIG. 3). Accordingly, the grasp part 200a that is grasped by a user and the exterior of the insertion unit 200b that comes in contact with positions other than a surgical site of a patient can maintain an insulating state.

In the present embodiment, an insulating unit 230 is formed by coating the exterior of the radio frequency supply unit 220 with silicon materials. In addition, the insulating unit 230 may be made of insulating materials, such as polytetrafluorethlene (PTFE) resin or parylene resin.

The front end of the radio frequency supply unit 220 is protruded from the front end of the insulating unit 230 in a specific length, thereby forming a radio frequency electrode 221. In the present embodiment, the radio frequency electrode 221 is protruded from the front end of the insulating unit in a length of 0.2 mm or more to 10 mm or less. Accordingly, radio frequency electronic energy can be supplied in the state in which a proper contact surface with tissue within the body has been secured.

Here, the front end of the radio frequency supply unit 220 corresponding to the radio frequency electrode 221 is subject to round processing. According to the results of experiments, it was found that a phenomenon in which an overcurrent flowed into an edge part of the radio frequency electrode 221 if an edge structure was formed in the radio frequency electrode 221 was generated. Accordingly, the radio frequency electrode 221 can be formed to have a curved surface through round processing in order to minimize the generation of an overcurrent.

In the present embodiment, the radio frequency supply unit 220 itself has been configured to form the body of the handpiece 200, but the handpiece 200 may be changed to have various structures. For example, the body of the handpiece may be made of non-conductive materials, and the radio frequency supply unit may be formed by indenting conductive metal into the body.

Furthermore, in the present embodiment, the front end of the body of the handpiece has been configured to form one radio frequency electrode, but this is only an example. The handpiece may have a variety of modifications, such as a modification in which a plurality of radio frequency electrodes is deployed in the outer circumferential direction of the hole.

Meanwhile, as shown in FIG. 3, the hole is formed within the body of the handpiece 200 in such a way as to penetrate the body. The hole forms a path along which laser light transferred from the light transfer unit 310 travels. The hole is connected up to the front end of the handpiece 200, so the laser light is radiated through the light radiation unit 210.

In the present embodiment, the light path and the light radiation unit 210 are formed by inserting an optical fiber 310 that forms the light transfer unit 310 into the hole of the handpiece. That is, the end of the optical fiber 310 forms the light radiation unit 210, so light is radiated to a surgical site through the end of the optical fiber 310.

Here, the end of the optical fiber 310 is configured to protrude from the front end of the radio frequency electrode in a specific length. In this case, when the optical fiber 310 incises tissue within the body at the front thereof, the radio frequency electrode 221 can supply radio frequency electronic energy to the incised part. In the present embodiment, the front end of the optical fiber 310 is installed in such a way as to be protruded from the front end of the radio frequency electrode in a length of 1 mm or more to 8 mm or less.

As described above, in the optical surgery apparatus in accordance with the present embodiment, the optical fiber 310 is inserted into and installed in the hole of the handpiece 200 so that light is radiated through the front end of the optical fiber 310. However, this is only an example, and the optical fiber may be detachably installed at the rear end of the handpiece.

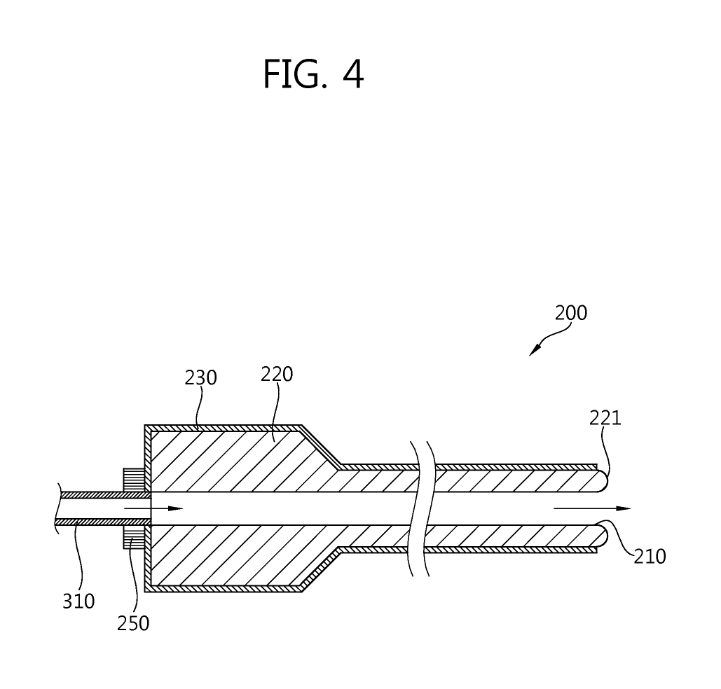

As shown in FIG. 4, the optical fiber 310 is connected and installed by an additional fastening member 250 that is provided at the rear end of the handpiece 200. The light path of the optical fiber 310 is disposed in the same axis as that of the hole of the handpiece 200. Here, the hole of the handpiece 200 may form a path along which light transferred along the optical fiber 310 travels, and the light radiation unit 210 may be formed at the front end of the hole so that light is radiated.

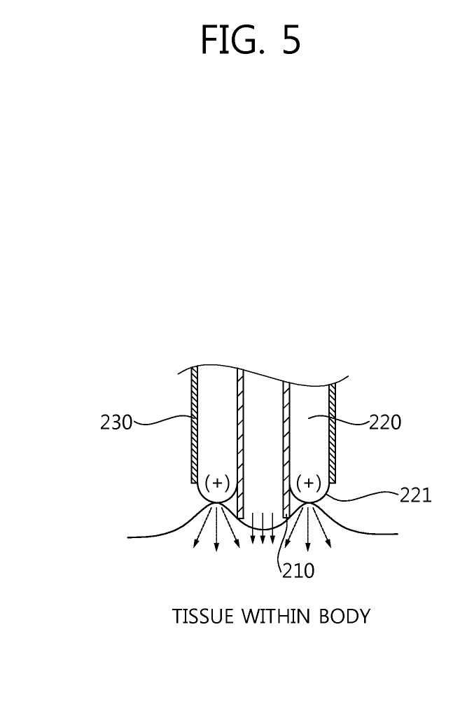

FIG. 5 is a cross-sectional view showing a state in which surgery is performed using the handpiece of FIG. 3. As shown in FIG. 5, the handpiece 200 of the optical surgery apparatus in accordance with the present embodiment can perform incision and hemostasis on tissue within the body in the state in which the handpiece 200 has approached the tissue within the body.

An incision operation is performed in such a way as to radiate incision light through the light radiation unit 210 formed at the end of the handpiece 200. As described above, laser light having a wavelength of 1444 nm is used as the incision light radiated through the light radiation unit 210. Here, the incision light consists of pulse waves having strong output, and thus the incision operation is performed in such a way as to strike tissue within the body with strong energy when the incision light is radiated.

A hemostasis operation is performed in such a way as to supply radio frequency electronic energy using the radio frequency electrode 221 formed at a portion adjacent to the light radiation unit 210. As described above, the radio frequency electrode 221 of the handpiece 200 is formed of a mono-polar type electrode and the external electrode 500 corresponding to the mono-polar type electrode is attached to the outside of the body, so the external electrode and the mono-polar type electrode form a circuit.

If the radio frequency electrode 221 form a (+) electrode as shown in FIG. 5, the external electrode 500 attached to the outside of the body forms a (−) electrode. On the contrary, if the radio frequency electrode 221 forms a (−) electrode, the external electrode 500 forms a (+) electrode, thereby forming a circuit.

As described above, radio frequency electronic energy supplied to the interior of the body is converted into heat energy by means of the radio frequency electrode 221 and the external electrode 500. Here, the heat energy supplied to the interior of the body is concentrated upon a portion where the electrodes 221 and 500 are formed. As a result, the heat energy is concentrically supplied to a portion adjacent to the radio frequency electrode 221 having an area that is relatively narrower than a wide surface area of the external electrode 500.

Accordingly, when tissue within the body is incised by incision light radiated from the light radiation unit 210, the incised part can be heated by radio frequency electronic energy that is supplied by the radio frequency electrode 221 provided at a position adjacent to the light radiation unit 210. Accordingly, hemostasis can be performed because the blood congeals.

Here, a surgical operation can be performed while stopping the flow of blood from the incised part in such a manner that the control unit 400 controls an operation of radiating light through the light radiation unit 210 and an operation of performing hemostasis through the radio frequency electrode 221 so that the operations are performed alternatively or simultaneously.

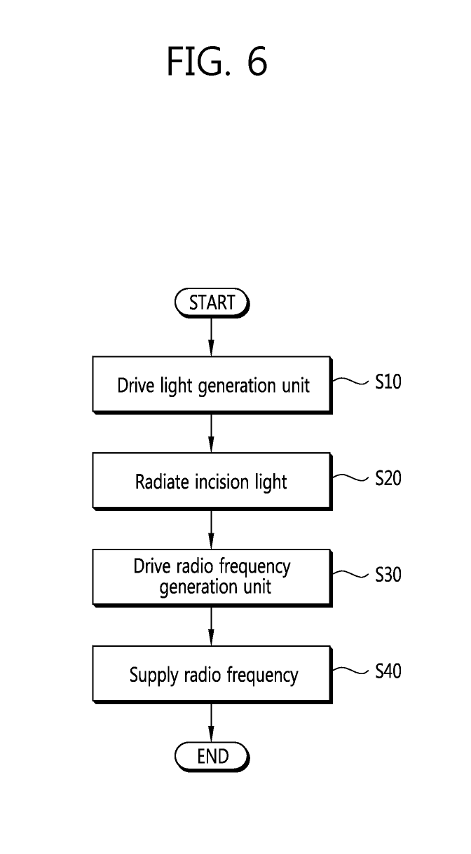

FIG. 6 is a flowchart illustrating a method of controlling the optical surgery apparatus of FIG. 1. Hereinafter, the method of controlling the optical surgery apparatus is described in more detail with reference to FIG. 6.

First, a user attaches the external electrode 500 of the optical surgery apparatus to the outside of the body of a patient. Furthermore, the user inserts the handpiece 200 in which the optical fiber 310 is installed into the body and then places the handpiece 200 in a surgical site that needs to be incised.

Furthermore, the control unit 400 generates incision light by driving the light generation unit 110 (S10). The incision light generated from the light generation unit 110 is radiated for a predetermined first time through the light radiation unit 210 of the handpiece 200 via the light transfer unit 310 of the cable 300 (S20). The incision light has high-output pulse waves and incises tissue within the body in such a manner that an energy impulse is applied to the tissue within the body when the incision light is radiated.

Meanwhile, the control unit 400 generates radio frequency electronic energy by driving the radio frequency generation unit 120 (S30). Here, the radio frequency generation unit 120 may be controlled so that it is started in the state in which the incision operation is paused or may be controlled so that it continues to generate the radio frequency electronic energy while the incision operation is performed.

The radio frequency electronic energy generated from the radio frequency generation unit 120 is supplied to the body for a predetermined second time by means of the radio frequency electrode 221 of the handpiece 200 and the external electrode 500 attached to the outside of the body (S40). At this time, the radio frequency electronic energy is concentrically supplied to the incised part adjacent to the radio frequency electrode 221. The supplied radio frequency electronic energy is converted into heat energy, thus stopping the flow of blood from the incised part.

The control unit 400 can control the incision operation and the hemostasis operation corresponding to the incision operation so that to perform the incision operation for the predetermined first time and to perform the hemostasis operation for the predetermined second time are repeatedly performed as one sequence. Here, the hemostasis operation may be controlled so that it is performed at a point of time at which the incision operation is finished, and the hemostasis operation and the incision operation may be controlled so that they are performed at the same time.

Additionally, the control unit 400 may perform the step of determining whether or not stopping the flow of blood from the incised part has been completed. This step can be directly determined by a user based on an image of a surgical site or may be directly determined by a sensor (not shown) attached to the handpiece or the control unit through the processing of a captured image.

If, as a result of the determination, it is determined that the hemostasis has not been completed, the control unit 400 can drive the radio frequency generation unit 120 so that radio frequency electronic energy is additionally supplied to a part whose hemostasis has not been completed. Hemostasis can be finished such an additional hemostasis process.

As described above, the optical surgery apparatus in accordance with the present embodiment can reduce the time taken for a surgical operation by performing an incision operation and a hemostasis operation using pieces of energy having different types and can perform a surgical operation in various ways by freely controlling an incision operation and a hemostasis operation in various patterns.

In the above-described embodiment, the optical surgery apparatus in which a mono-polar type electrode is provided in the handpiece and an electrode corresponding to the mono-polar type electrode is installed outside the optical surgery apparatus and the method of controlling the same have been illustrated. However, the above-described construction of the optical surgery apparatus is only one example, and the optical surgery apparatus can be changed and implemented to have various structures.

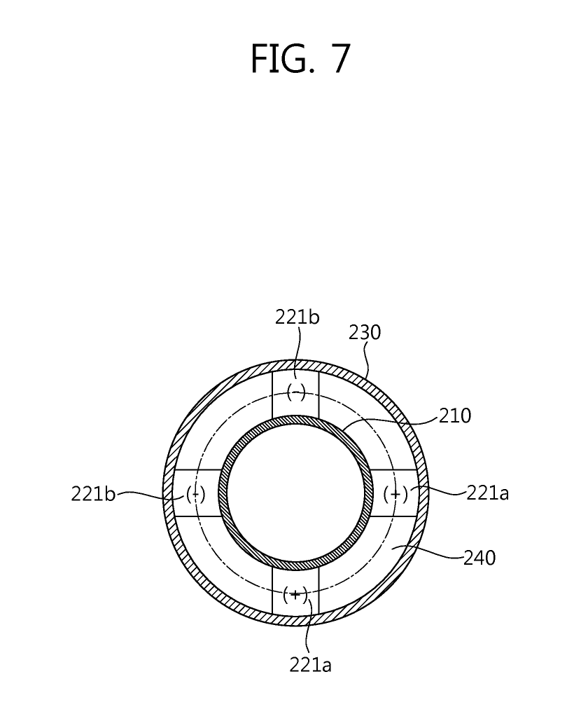

FIG. 7 is a front view showing the end of the handpiece of an optical surgery apparatus in accordance with a second embodiment of the present invention.

In the handpiece of the above-described embodiment, the radio frequency supply unit is formed of one SUS member, thus forming the body of the handpiece. In the present embodiment, the radio frequency supply unit is formed in such a manner that conductive metal is pressed in the body. Accordingly, a plurality of radio frequency electrodes 221a and 221b is formed at the front end of the radio frequency supply unit.

Some of the radio frequency electrodes 221a and 221b can form (+) electrodes 221a, and the remaining frequency electrodes can form (−) electrodes 221b. That is, in the above-described embodiment, the radio frequency electrode having a mono-polar type is formed in the handpiece, whereas in the present embodiment, the radio frequency electrodes 221a and 221b having a bipolar type can be formed in the handpiece 200.

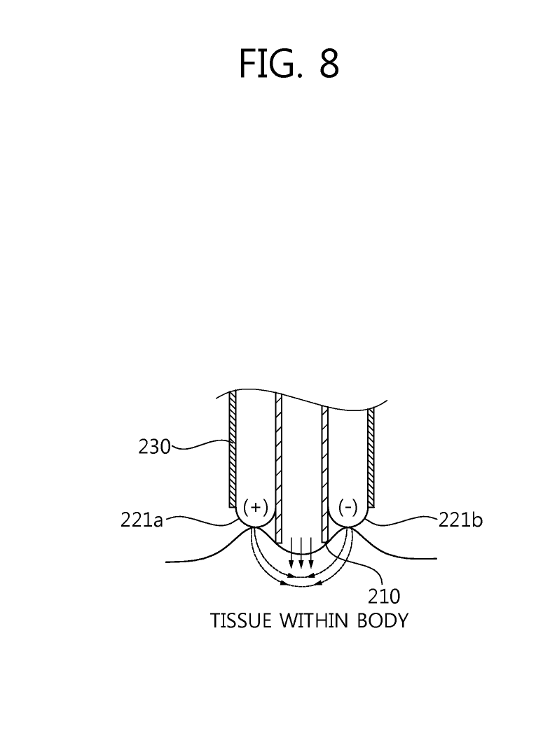

FIG. 8 is a cross-sectional view showing a state in which surgery is performed using the handpiece of FIG. 7. In the above-described embodiment, the radio frequency electronic energy is supplied by means of the radio frequency electrode placed in a surgical site and the external electrode attached to the outside of the body, whereas in the present embodiment, the radio frequency electronic energy is supplied by means of the (+) electrodes 221a and the (−) electrodes 221b placed in a surgical site. Accordingly, the radio frequency electronic energy can be supplied to only parts adjacent to the surgical site.

As described above, the optical surgery apparatus in accordance with the present invention can be configured in various ways by changing the location of electrodes from which a radio frequency is supplied and can be designed and changed in various ways depending on a surgical site and contents of a surgical operation in addition to the above-described embodiment.

In the above-described first and second embodiments, tissue within the body is incised using light energy, and the flow of blood from the incised part is stopped using radio frequency electronic energy. In contrast, in the followings embodiments, tissue within the body is incised using a plurality of pieces of light having different wavelengths and the incised part from the flow of blood is stopped.

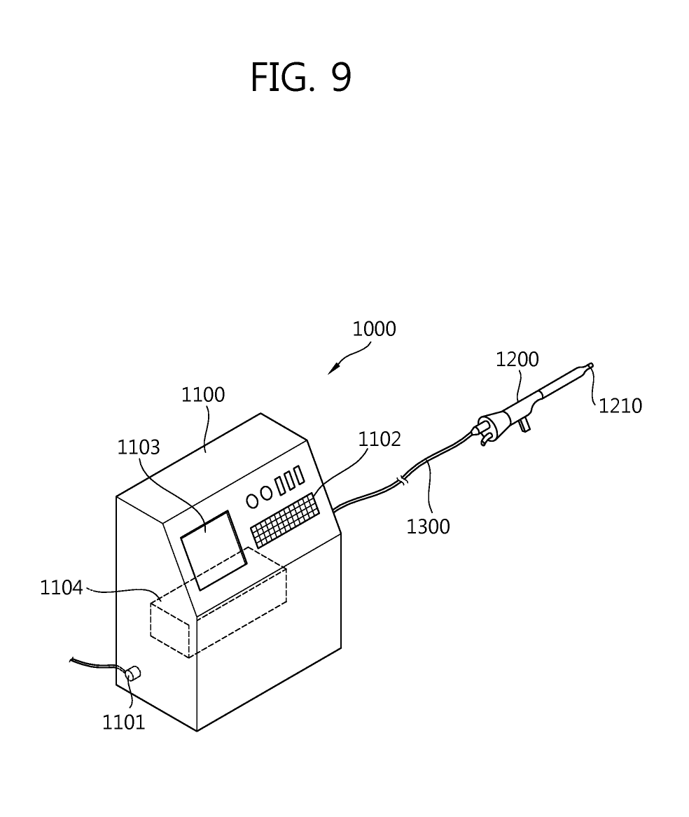

Hereinafter, optical surgery apparatuses in accordance with a third embodiment and a fourth embodiment of the present invention are in detail described with reference to FIGS. 9 to 14.

FIG. 9 is a perspective view showing an optical surgery apparatus in accordance with a third embodiment of the present invention. As shown in FIG. 9, the optical surgery apparatus 1000 in accordance with the present embodiment includes a main body 1100, a handpiece 1200, and a cable 1300 coupling the main body 1100 and the handpiece 1200.

The main body 1100 is equipped with a power supply unit 1101 capable of being supplied with a power source from the outside. A control panel 1102 for manipulating contents of driving of the optical surgery apparatus 1000 and a display 1103 for displaying the contents of driving to a user are installed in the exterior of the main body. Furthermore, a light generation unit 1104 for generating light for surgical operations is included within the main body 1100.

The end of the handpiece 1200 has a pointed form, such as a needle, so that the end can be inserted into the body when performing a surgical operation. A light radiation unit 1210 is formed at the end of the handpiece 1200. Accordingly, when performing a surgical operation, the end of the handpiece 1200 is placed at a surgical site, and the surgical operation is performed by radiating light through the light radiation unit 1210.

A light path along which light can travel to the light radiation unit 1210 is formed within the handpiece 1200. In addition, various devices for an effective surgical operation can be embedded and installed in the handpiece 1200. In the concrete, although not shown, a lighting signal line, an image signal line, etc. for capturing an image of a surgical site can be embedded and installed in the handpiece. Alternatively, a fluid passage can be provided within the handpiece so that water or air can be sprayed toward a surgical site.

Such a handpiece 1200 is configured to be grasped so that a user can perform a surgical operation while changing the location of the surgical operation. A manipulation unit 1220 that enables a user to easily manipulate contents (e.g., light radiation or water spray) of a surgical operation can be provided outside the handpiece 1200.

In the present embodiment, the handpiece 1200 and various elements embedded within the handpiece 1200 are integrally formed and are configured to be driven by the manipulation unit of the handpiece 1200, but this is only an example. In addition, various elements may be separately provided so that a surgical operation is performed by selectively inserting the various elements into the handpiece during the surgical operation.

Meanwhile, the cable 1300 is formed between the main body 1100 and the handpiece 1200. The cable 1300 includes a light transfer unit 1310 along which light generated from the light generation unit 1104 of the main body 1100 can travel to the light radiation unit 1210. Such a light transfer unit 1310 includes one or a plurality of optical fibers. In addition, a signal line 1320 for transferring various signals between the main body 1100 and the handpiece 1200, a fluid passage (not shown) along which a fluid flows, etc. can be embedded and installed in the cable.

Here, the optical fiber may be connected with one end of the handpiece so that the optical axis of the optical fiber is connected with the light path within the handpiece 1200. Alternatively, one end of the optical fiber may penetrate the handpiece 1200 so that the end of the optical fiber forms the light radiation unit 1210 at the end of the handpiece 1200.

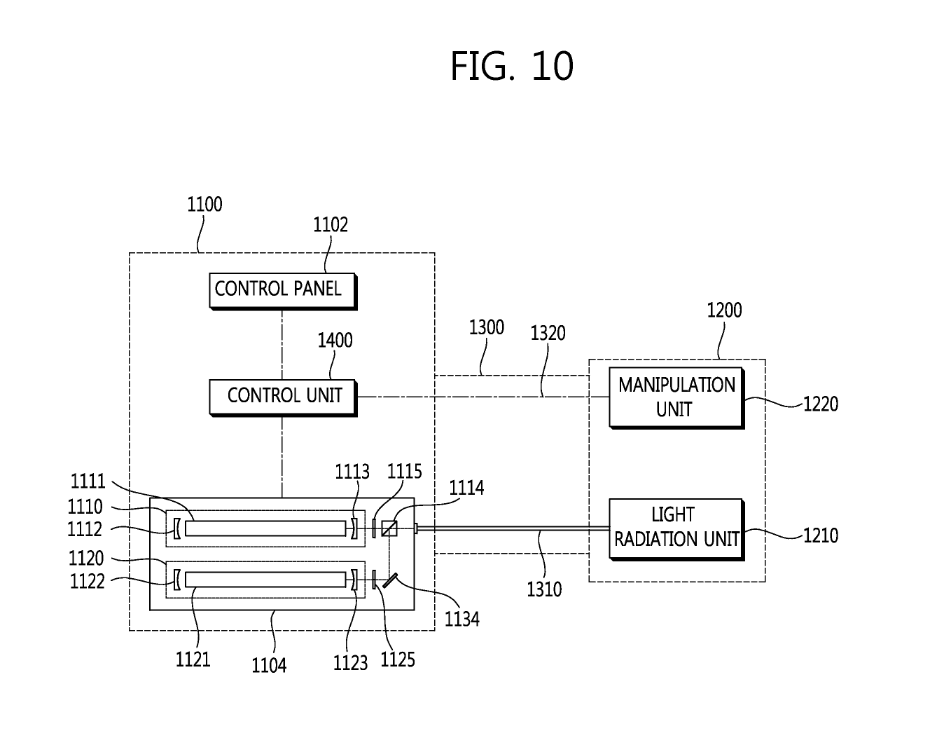

FIG. 10 is a block diagram schematically showing the construction of the optical surgery apparatus of FIG. 9. Hereinafter, the construction of the optical surgery apparatus in accordance with the present embodiment is described in more detail with reference to FIG. 10.

As shown in FIG. 10, the light generation unit 1104 includes a first light generator 1110 and a second light generator 1120. The first light generator 1110 and the second light generator 1120 are formed of resonators capable of oscillating lasers. Laser media 1111 and 1121 are included in the respective resonators. Total reflection mirrors 1112 and 1122 and partial reflection mirrors 1113 and 1123 are provided at both ends of the respective laser media 1111 and 1121. In addition, various types of optical members (not shown) can be installed. Accordingly, the laser medium oscillates light by means of an exciting medium, such as a flash lamp (not shown), and the oscillating light is amplified while going and coming back within the resonator, thereby generating laser light.

In the present embodiment, the first light generator 1110 is an incision energy source, and it generates light for incision, which can incise tissue within the body. In the concrete, the first light generator includes Nd:YAG as the laser medium and generates first light having a wavelength of 1444 nm.

In contrast, the second light generator 1120 is a hemostasis energy source, and it generates light for hemostasis, which can stop the flow of blood from an incised tissue. In the concrete, the second light generator includes Nd:YAG as the laser medium, and it generates second light having a wavelength of 1064 nm.

As described above, the first light generator 1110 and the second light generator 1120 are configured to generate pieces of light having different wavelengths. Accordingly, since the first light and the second light have different absorption characteristics in tissue within the body, the first light can be used to supply energy for incision to tissue within the body, and the second light can be used to supply energy for hemostasis to tissue within the body.

In the present embodiment, the light generation unit 1104 is configured to generate light having a wavelength of 1444 nm and light having a wavelength of 1064 nm so that a surgical operation for incising tissue and stopping the flow of blood from the incised tissue is performed, but the present invention is not limited to the wavelengths of the pieces of light. Pieces of light having various wavelengths can be used depending on contents of a surgical operation.

Furthermore, in the present embodiment, the light generator is formed of the resonator for oscillating light using a flash lamp. It is however to be noted that various light generators, such as a gas laser and a semiconductor laser, can be applied to the light generator.

Meanwhile, the light transfer unit 1310 that forms a light path is installed on one side of the light generation unit 1104. Furthermore, various types of optical members can be provided between the light generation unit 1104 and the light transfer unit 1310. Accordingly, the first light and the second light generated from the first light generator 1110 and the second light generator 1120 can be transferred to the light radiation unit 1210 via the light transfer unit 1310.

In the concrete, as shown in FIG. 10, a splitter 1114 for selectively transmitting or reflecting light depending on a wavelength characteristic is provided on one side of the first light generator 1110. Here, the splitter 1114 is configured to transmit the wavelength band of the first light, and the first light generated from the first light generator 1110 passes through the splitter and enters the light transfer unit 1310.

In contrast, a reflection mirror 1124 can be provided on one side of the second light generator 1120. Accordingly, the second light generated from the second light generator 1120 is reflected by the reflection mirror 1124, and the reflected second light enters the splitter 1114. Here, the splitter 1114 is configured to reflect the wavelength band of the second light, and thus the second light is reflected by the splitter 1114, thus entering the light transfer unit 1310.

That is, the first light and the second light generated from the first and the second light generators 1110 and 1120, respectively, travel along the same light path by means of the splitter 1114. Accordingly, the light radiation unit 1210 radiates the first light or the second light, provided along the light transfer unit 1310, to a surgical site so that a surgical operation can be performed.

Meanwhile, the optical surgery apparatus 1000 in accordance with the present embodiment includes a control unit 1400 for controlling the elements. The control unit 1400 can control contents of driving of the optical surgery apparatus 1000 based on contents set by a user through the control panel 1102, contents manipulated by a user through the manipulation unit 1220 that is provided in the handpiece 1200, or conditions stored in its memory (not shown).

For example, the control unit 1400 can control the operations of the first light generator 1110 and the second light generator 1120. The operation of the light generator is performed differently from the operation of the flash lamp for exciting the resonator. Accordingly, the control unit 1400 can control whether or not to generate light, the output amount of light, the frequency of light, a pulse waveform of light, etc. by controlling circuits connected with the flash lamps.

Furthermore, the control unit 1400 can control the first light generator 1110 and the second light generator 1120 so that the first light and the second light generated from the first light generator 1110 and the second light generator 1120 are selectively radiated through the light radiation unit. For example, the control unit can perform control so that only the first light or the second light is radiated through the light radiation unit by selectively driving the first light generator and the second light generator. Alternatively, the control unit 1400 can independently control shutters 1115 and 1125 installed in the respective light paths of the first light and the second light so that the first light and the second light are selectively radiated.

In addition, if the optical surgery apparatus 1000 includes an additional photographing unit, an additional lighting unit, an additional fluid spray unit, etc., the control unit 1400 can control the photographing unit, the lighting unit, the fluid spray unit, etc.

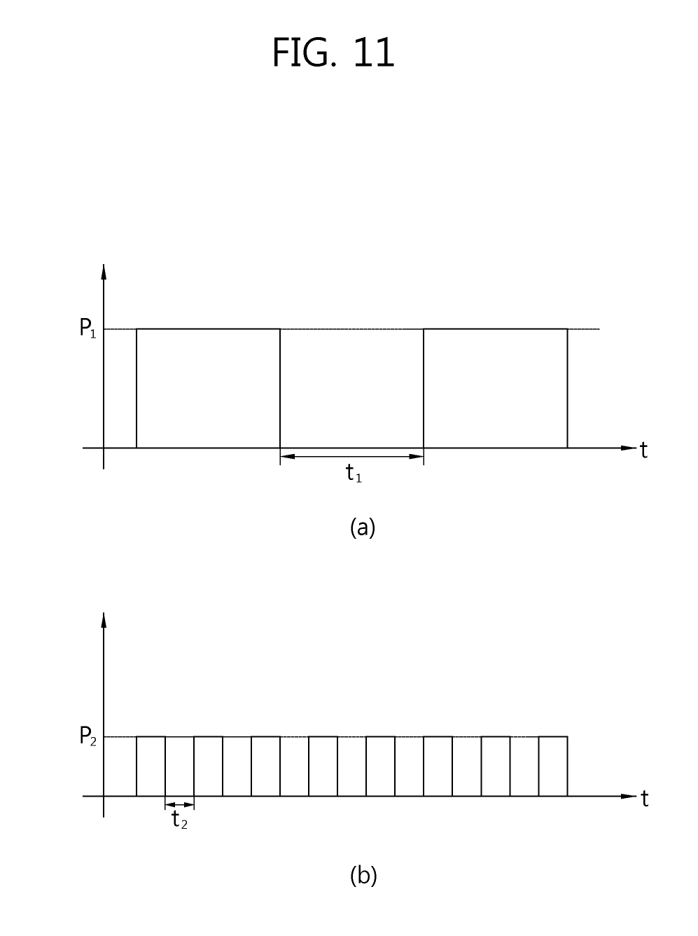

FIG. 11 is a graph showing pulse forms of the first light and the second light. Hereinafter, the first light and the second light that are radiated through the light radiation unit are described in detail with reference to FIG. 11.

As described above, the first light is used as incision light capable of incising tissue within the body when performing a surgical operation. The first light can incise a corresponding location by radiating heat energy to a local part when it is radiated to tissue within the body.

Meanwhile, the second light is used as hemostasis light for stopping the flow of blood from an incised part of tissue within the body when performing a surgical operation. The second light can stop the flow of blood from tissue within the body by continuously supplying relatively small heat energy to an incised part and parts adjacent to the incised part.

In the concrete, laser pulse light that is intermitted in a specific period is used as each of the first light and the second light. Here, as described above, laser light having a wavelength of 1444 nm is used as the first light that is incision light, and laser light having a wavelength of 1064 nm is used as the second light that is hemostasis light.

When the light having a wavelength of 1064 nm is radiated to tissue within the body, heat is easily spread up to parts to which the light has not been radiated, whereas when the light having a wavelength of 1444 nm is radiated, heat is less spread up to parts to which the light has not been radiated as compared with the light having a wavelength of 1064 nm.

Accordingly, the first light having a wavelength of 1444 nm can incise only a target part while minimizing a thermal influence on parts adjacent to the target part when incising tissue. Furthermore, when the second light having a wavelength of 1064 nm is radiated, heat is easily spread. Accordingly, heat energy can be provided up to parts adjacent to an incised part, thereby being capable of stopping the flow of blood.

Meanwhile, the incision light incises tissue in such a way as to burn the tissue by supplying great heat energy to tissue within the body or incises tissue in such a way as to strike the tissue by instantly supplying strong energy to tissue within the body. In contrast, the hemostasis light stops the flow of blood in such a way as to congeal the blood by providing relatively small heat energy to tissue within the body. Accordingly, as shown in FIG. 11, output P₁ of the first light radiated from the light radiation unit 1210 is greater than output P₂ of the second light.

Here, each of the first light and the second light has a pulse waveform that is periodically intermitted for a specific time as described above. Here, the pulse period of the first light is longer than the pulse period of the second light, and duration t₁ during which the first light is intermitted in one period is formed to be longer than duration t₂ during which the second light is intermitted in one period.

The first light, that is, incision light, incises tissue by concentrically providing energy having strong output to tissue within the body. Here, the duration t₁ from the time when n^{th} light is intermitted to the time when (n+1)^{th} light is radiated can exceed 0.15 μs so that heat energy can be prevented from being accumulated on tissue within the body as the incision light is repeatedly radiated.

In contrast, the second light, that is, hemostasis light, stops the flow of blood from tissue by continuously supplying tissue within the body with energy having relatively weak output. Accordingly, when radiating the second light, duration from the time when m^{th} light is intermitted to the time when (m+1)^{th} light is radiated can maintain 0.15 μs or less. In this case, if the intermittence time of the second light is 0.15 μs or less, the (m+1)^{th} light is radiated before heat energy provided to the tissue within the body disappears when the m^{th} light is radiated. Accordingly, a hemostasis effect is improved as if light having continuous waves is radiated.

The optical surgery apparatus 1000 in accordance with the present embodiment radiates laser light having a frequency of 40 Hz with output of 1 J when radiating the first light and radiates laser light having a frequency of 100 Hz with output of 0.2 J when radiating the second light. Furthermore, when radiating the first light, an intermittence time as per one period exceeds 0.15 μs, and when radiating the second light, an intermittence time as per one period has 0.15 μs or less. It is however to be noted that the outputs and frequencies are only examples and the outputs and frequencies can be changed depending on a surgical site and treatment purposes.

Meanwhile, the second light generator 1120 of the present embodiment has been configured using the resonator for generating a pulse type laser, but the present invention is not limited thereto. The second light generator can be configured using a light source for generating continuous light in order to achieve the second light having excellent hemostasis performance. Furthermore, since light having low output is used when performing hemostasis, the second light generator can be configured using a laser diode for generating a laser having low output.

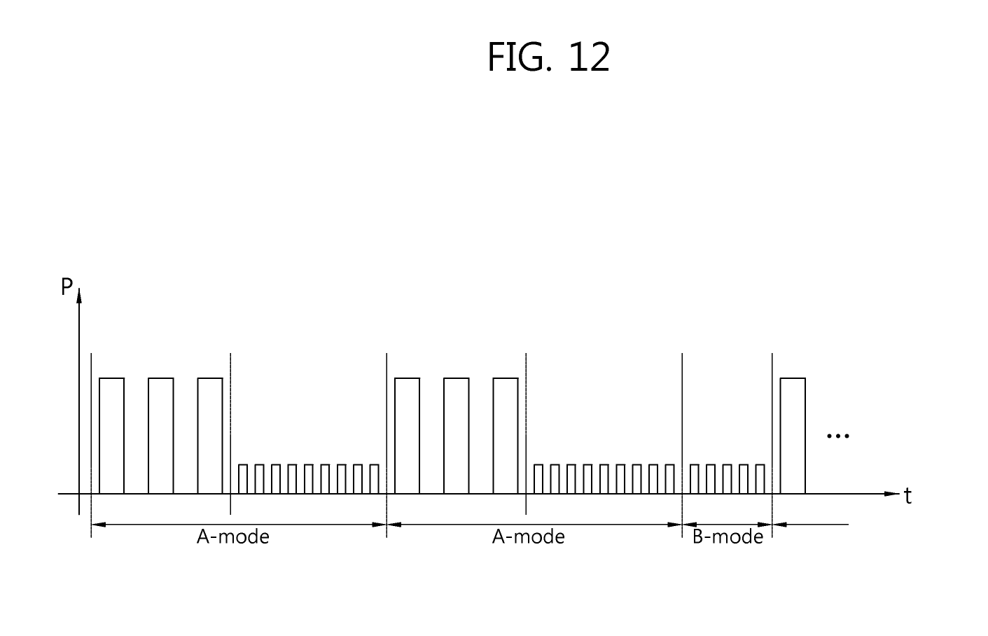

FIG. 12 is a graph showing a radiation pattern of light that is radiated from the light radiation unit of FIG. 11.

The optical surgery apparatus 1000 in accordance with the present invention can control a pattern of light radiated through the light radiation unit 1210 by means of control of the control unit 1400.

For example, as shown in FIG. 12, control can be performed so that light is radiated using the radiation of the first light for the first time and the subsequent radiation of the second light for the second time as one sequence (refer to a mode A of FIG. 12).

In the concrete, the control unit 1400 drives the first light generator 1110 and opens the shutter 1115 provided in the light path of the first light so that the first light is radiated. At this time, the shutter 1125 in the light path of the second light is closed, so the radiation of the second light through the light radiation unit 1210 is cut off. When the first light radiation time is finished, the control unit drives the second light generator 1120 and opens the shutter 1125 provided in the light path of the second light so that the second light is radiated through the light radiation unit 1210. At this time, the shutter 1115 provided in the light path of the first light is closed, so the radiation of the first light is cut off. In this case, when performing a surgical operation, an operation for incising tissue within the body while the first light is radiated and then stopping the flow of blood from the incised part by radiating the second light can be performed as one sequence.

FIG. 12 shows that such a sequence is continuously performed twice. However, such a sequence can be repeatedly performed continuously by means of control of the control unit 1400, and a design is possible so that one sequence is performed whenever a user manipulates the manipulation unit.

Meanwhile, if continuous hemostasis operations after incision are repeated as one operation as in the mode A of FIG. 12, hemostasis may not be properly performed when radiating the second light. Accordingly, the control unit 1400 may perform control in a mode in which only the second light is radiated for a specific time so that additional hemostasis can be performed (refer to a mode B of FIG. 12). Accordingly, hemostasis can be completed by radiating the second light in the mode B, and the mode B can switch to the mode A again in order to perform incision and hemostasis operations.

FIG. 12 shows that when the mode A operates, the first light is radiated for 3 pulses and the second light is radiated for 9 pulses and when the mode B operates, the second light is radiated for 5 pulses. However, this is only an example, for convenience of description, and duration for which the first light or the second light is radiated in each mode can be controlled in various ways.

Furthermore, FIG. 12 illustrates only the two control modes, but a radiation pattern of light can be modified in various ways according to user needs.

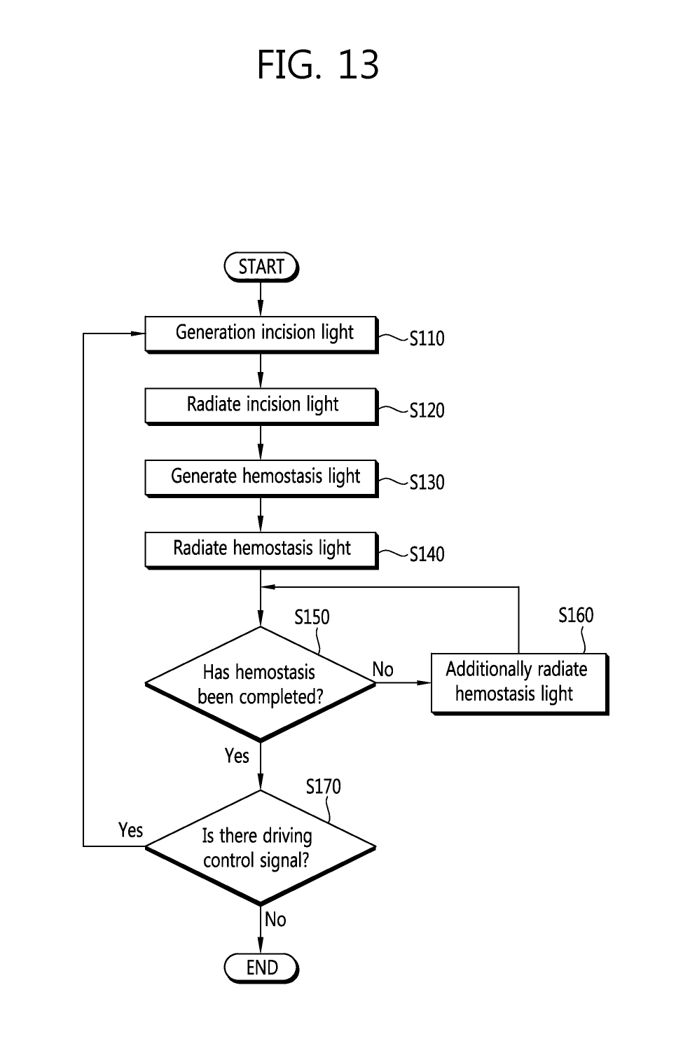

FIG. 13 is a flowchart illustrating a method of controlling the optical surgery apparatus of FIG. 9. Hereinafter, the method of controlling the optical surgery apparatus is described in more detail with reference to FIG. 13.

First, a user installs the light radiation unit 1210 of the optical surgery apparatus 1000 in the handpiece 1200, inserts the light radiation unit 1210 into the body, and places the light radiation unit 1210 in a surgical site that needs to be incised.

Next, the control unit 1400 drives the first light generator 1110 so that the first light, that is, incision light, is generated (S110). The incision light generated from the first light generator 1110 travels along the light transfer unit 1310 and incises tissue within the body by illuminating a surgical site through the light radiation unit 1210 (S120). At this time, the shutter 1125 disposed in the light path of the second light remains closed.

Meanwhile, the control unit 1400 drives the second light generator 1120 so that the second light, that is, hemostasis light, is generated (S130). Here, the second light generator 1120 may be controlled so that it starts being driven at a point of time at which the radiation of the first light is terminated and then generates the second light or may be controlled so that it continues to drive and generate the second light while the first light is radiated.

After the step (S120) of radiating the first light is terminated, the second light generated from the second light generator 1120 travels along the light transfer unit 1310, and the second light is radiated to the surgical site through the light radiation unit 1210 (S140). At this time, the shutter 1125 placed on the light path of the second light is opened, and the shutter 1115 placed on the light path of the first light is opened. Accordingly, one sequence in which hemostasis is performed and tissue is incised while the second light is radiated through the light radiation unit 1210 is performed (refer to the mode A of FIG. 12).

Next, whether or not stopping the flow of blood from the incised part has been completed is determined (S150). Such a determination may be directly made by a user based on an image of the surgical site or may be directly made by a sensor (not shown) attached to the handpiece 1200 or by the control unit 1400 through the processing of a captured image.

If, as a result of the determination, it is determined that stopping the flow of blood from the incised part has not been completed, the control unit 1400 controls the second light so that the second light is additionally radiated (S160, mode B of FIG. 12). Accordingly, the hemostasis can be finished through the additional hemostasis process.

In contrast, if, as a result of the determination, it is determined that stopping the flow of blood from the incised part has been completed, the control unit 1400 determines whether or not there is an additional driving control signal. If, as a result of the determination, it is determined that there is a driving control signal, the control unit 1400 can repeatedly perform the mode A. If, as a result of the determination, it is determined that there is no driving control signal, the control unit 1400 can terminate the process.

The optical surgery apparatus capable of performing various surgical operations using two different wavelengths and the method of controlling the same have been described above, but the above-described construction of the optical surgical apparatus can be changed and implemented to have various structures, for example. An example of a modified embodiment is described below.

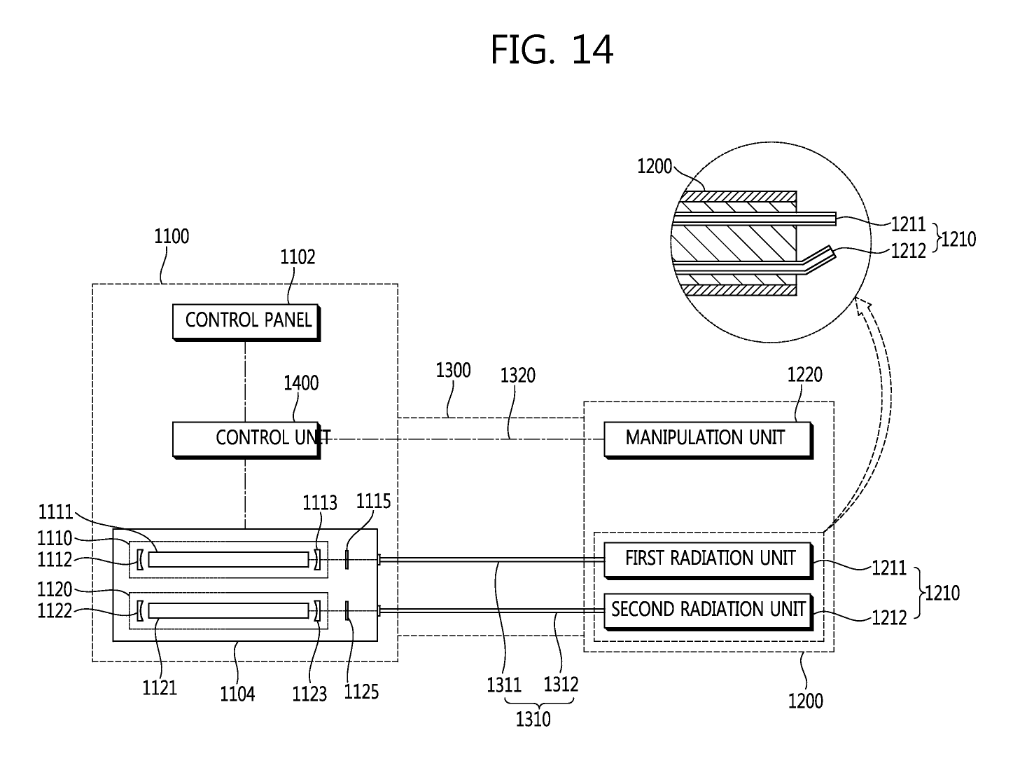

FIG. 14 is a block diagram schematically showing the structure of an optical surgery apparatus in accordance with a fourth embodiment of the present invention.

In the above-described third embodiment, the splitter and the reflection mirror are provided on one side of the first light generator and the second light generator. Accordingly, the first light and the second light are configured to share one light path and are configured to be radiated through the same light radiation unit.

In contrast, as shown in FIG. 14, the light transfer unit 1310 of the present embodiment is configured to include a first path 1311 along which the first light travels and a second path 1312 along which the second light travels. The first path and the second path are formed of separate optical fibers. The first path 1311 is formed on one side of the first light generator 1110, thus forming a path along which the first light travels to the light radiation unit 1210. The second path 1312 is formed on one side of the second light generator 1120, thus forming a path along which the second light travels.

Furthermore, the light radiation unit 1210 includes a first radiation unit 1211 and a second radiation unit 1212. The first radiation unit 1211 radiates the first light that travels through the first path 1311, and the second radiation unit 1212 radiates the second light that travels the second path 1312.

As described above, unlike in the above-described third embodiment, the optical surgery apparatus can be configured so that paths along which the first light and the second light travel and the locations to which the first light and the second light are radiated are separately provided. In addition, the optical surgery apparatus can be designed and changed in various ways.

Furthermore, in the present embodiment, the optical surgery apparatus for radiating two pieces of light has been configured, but the optical surgery apparatus can be configured to selectively three or more pieces of light.

1. An optical surgery apparatus, comprising:
an incision energy source for generating energy for incision for incising a surgical site;
a hemostasis energy source formed separately from the incision energy source, for generating energy for hemostasis for stopping a flow of blood from the incised surgical site;
a handpiece connected with the incision energy source and the hemostasis energy source, for providing the energy for incision and the energy for hemostasis to the surgical site; and
a control unit for controlling the energy for incision and the energy for hemostasis supplied through the handpiece.

2. The optical surgery apparatus of claim 1, wherein:
the incision energy source generates light for incision, and
the hemostasis energy source generates radio frequency electronic energy for hemostasis.

3. The optical surgery apparatus of claim 1, wherein:
the incision energy source generates light for incision, and
the hemostasis energy source generates light for hemostasis having a different wavelength from the light for incision.

4. An optical surgery apparatus, comprising:
a light radiation unit for radiating light, generated from a light generation unit, to a surgical site within a body;
a radio frequency supply unit installed adjacent to an end of the light radiation unit, for providing radio frequency electronic energy to a part to which the light is radiated;
an insulating unit formed to surround part of the radio frequency supply unit; and
a control unit for controlling driving of the light radiation unit and the radio frequency supply unit.

5. The optical surgery apparatus of claim 4, wherein:
the light radiation unit radiates incision light for incising tissue within the body, and
the radio frequency supply unit supplies the radio frequency electronic energy for stopping a flow of blood from the incised tissue.

6. The optical surgery apparatus of claim 5, wherein:
the radio frequency supply unit comprises a thin pipe in which a hole is formed in a length direction, and
the light radiation unit is inserted into and installed in the hole.

7. The optical surgery apparatus of claim 5, wherein:
a mono-polar type radio frequency electrode is formed at a front end of the radio frequency supply unit, and
the optical surgery apparatus further comprises an external electrode having a different polarity from the radio frequency electrode and formed to be attached to a surface of the body.

8. The optical surgery apparatus of claim 5, wherein:
a plurality of radio frequency electrodes is formed at a front end of the radio frequency supply unit, and
some of the plurality of radio frequency electrodes form positive electrodes and remaining radio frequency electrodes form negative electrodes.

9. The optical surgery apparatus of claim 5, wherein a front end of the radio frequency supply unit is subject to round processing.

10. The optical surgery apparatus of claim 5, wherein a front end of the light radiation unit is protruded from a front end of the radio frequency supply unit in a length of 1 cm or less.

11. A method of controlling an optical surgery apparatus, comprising steps of:
generating incision light capable of incising tissue within a body by driving a light generation unit;
radiating the incision light to the tissue within the body through a light radiation unit;
generating radio frequency electronic energy by driving a radio frequency generation unit; and
supplying the radio frequency electronic energy to the tissue within the body through an radio frequency electrode installed adjacent to the light radiation unit.

12. An optical surgery apparatus, comprising:
a light generation unit for generating incision light and hemostasis light having a different wavelength from the incision light;
a light radiation unit for selectively radiating the incision light and the hemostasis light, generated from the light generation unit, to a surgical site; and
a control unit for controlling a radiation pattern of the light radiated through the light radiation unit.

13. The optical surgery apparatus of claim 12, wherein the control unit controls the light radiation unit so that the incision light having greater output than the hemostasis light is radiated.

14. The optical surgery apparatus of claim 13, wherein:
each of the incision light and the hemostasis light is pulse light that is periodically intermitted for a predetermined time, and
the hemostasis light is intermitted for a time shorter than a time of the incision light.

15. The optical surgery apparatus of claim 14, wherein:
the incision light is the pulse light periodically intermitted for a time exceeding 150 μs, and
the hemostasis light is the pulse light periodically intermitted for a time of 150 μs or less.

16. The optical surgery apparatus of claim 13, wherein:
the incision light is pulse light intermitted in a predetermined period, and
the hemostasis light is continuous light.

17. The optical surgery apparatus of claim 13, wherein:
the incision light is light having a wavelength of 1444 nm, and
the hemostasis light is light having a wavelength of 1064 nm.

18. The optical surgery apparatus of claim 13, wherein the control unit controls the light radiation unit so that a radiation of the incision light for a first time and a subsequent radiation of the hemostasis light for a second time are performed as one sequence.

19. A method of controlling an optical surgery apparatus, comprising steps of:
generating light for incision by driving a light generation unit;
radiating the light for incision to a surgical site through a light radiation unit;
generating light for hemostasis having a different wavelength from the light for incision through the light generation unit; and
radiating the light for hemostasis to the surgical site to which the light for incision has been radiated through the light radiation unit.

20. The method of claim 19, wherein the light for incision having greater output than the light for hemostasis is radiated through the light radiation unit.
